(19) United States
(12) Patent Application Publication
(10) Pub. No.: US 2007/0068805 A1
(43) Pub. Date: Mar. 29, 2007

(54) Operating circuit for a biosensor arrangement

(76) Inventors: Christian Paulus, Weilheim (DE); Meinrad Schienle, Ottobrunn (DE)

Correspondence Address: SCHIFF HARDIN, LLP;PATENT DEPARTMENT, 6600 SEARS TOWER, CHICAGO, IL, 60606-6473, US

(21) Appl. No.: 10/551,865

(22) PCT Filed: Apr. 2, 2004
(86) PCT No.: PCT/DE04/00690

§ 371 Date: Sep. 13, 2006

(30) Foreign Application Priority Data
Apr. 2, 2003 (DE) 10315080.3

(51) Int. Cl. G01N 33/487

(52) U.S. Cl. 204/403.01

(57) ABSTRACT

The invention relates to a sensor arrangement and sensor array with a sensor arrangement for the detection of particles possibly contained in an electrolytic analyte, comprising a working electrode which may be electrically coupled to the electrolytic analyte, with immobilized trap molecules such that in the presence of the electrolytic analyte containing the particles for detection, sensor events occur at the working electrode of the sensor arrangement. Furthermore, an auxiliary electrode which may be electrically coupled to the electrolytic analyte is provided and an operating circuit coupled to the working electrode, embodied such as to maintain an essentially constant potential difference between the working electrode and the auxiliary electrode. The sensor also comprises a device, embodied to maintain an essentially constant ratio between the current flowing to the working electrode and the current flowing to the auxiliary electrode.

### BACKGROUND

The invention concerns a sensor arrangement and a sensor array.

Biosensors for the detection of macromolecular biomolecules are increasingly gaining importance. Known DNA sensors are described by Hofmann, F., et al. “Passive DNA Sensor with Gold Electrodes Fabricated in a CMOS Backend Process” Proc. ESSDERC 2002, Digest of Tech. Papers, pp. 487-90; and Thewes, R., et al. “Sensor Arrays for Fully Electronic DNA Detection on CMOS”, ISSCC, Digest of Tech. Papers, 2002, pp. 350-51.

An important sensor type, particularly in completely electronic DNA sensor chips, is what is known as redox cycling. The bases of redox cycling are described in Hintsche. R., et al. “Microelectrode arrays and application to biosensing devices”, Biosensors & Bioelectronics, Vol. 9, pp. 697-705, 1994; and Hintsche, R., et al. “Microbiosensors Using Electrodes Made in Si-Technology”, Frontiers in Biosensorics, Fundamental Aspects, F. W. Scheller, et al. (eds.), Dirk Hauser Verlag, Basel, pp 267-83, 1997.

In redox cycling, macromolecular biopolymers on surfaces are electronically verified via detection of electrical currents caused by way of redox-active substances.

FIGS. 1A and 1B show a redox cycling sensor arrangement according to the prior art.

The redox cycling sensor arrangement 100 comprises two gold electrodes 101, 102 that are formed on a substrate 103. DNA capture molecules 104 with a predetermined sequence are immobilized on each gold electrode 101, 102. For example, the immobilization ensues according to what is known as gold-sulfur coupling. An analyte 105 to be examined is also brought into the redox cycling sensor arrangement 100. The analyte can, for example, be an electrolytic solution with different single-stranded DNA molecules.

If first DNA half-strands 106 with a sequence that is not complementary to the sequence of the DNA capture molecules 104 are contained in the analyte 105, these first DNA half-strands 106 do not hybridize with the DNA capture molecules 104 (see FIG. 1A). This situation is referred to as a “mismatch”. In contrast to this, if second DNA half-strands 107 with a sequence that is complementary to the sequence of the DNA capture molecules are contained in the analyte 105, these second DNA half-strands 107 hybridize with the DNA capture molecules 104. This situation is referred to as a “match”. Expressed otherwise, a DNA half-strand 106, 107 of a predetermined sequence is only respectively in the position to selectively hybridize with a very specific DNA half-strand, namely with the DNA half-strand with the sequence complementary to the respective capture molecule.

As shown in FIG. 1B, the second DNA half-strands 107 to be detected comprise a marker 108. After the hybridization of the second DNA half-strands 107 to be detected with the DNA capture molecules 104, given the presence of suitable secondary molecules 109, a cycle of oxidations and reductions of components of the secondary molecules 107 is initiated by way of the marker 108, which cycle leads to the formation of reduced molecules 110 or oxidized molecules 111 under interaction with the gold electrodes 101, 102. The cycle of oxidations and reductions leads to an electrical loop current that enables a detection of the second DNA half-strand 107.

A basic requirement for the functionality of a redox cycling sensor arrangement is the exact adjustment capability of the electrical potentials at the electrodes 101, 102. An oxidizing electrical potential is necessary at the first gold electrode 101, which can also be designated as a generator electrode. A reducing electrical potential is required at the second gold electrode 102, which can also be designated as a collector electrode.

FIG. 2 shows a diagram 200 along whose abscissa 201 an electrical potential is plotted, in contrast to which an electrical current is plotted along the ordinate 202 of diagram 200. A cyclic voltammogram for para-aminophenol is shown in the diagram 200. Para-aminophenol is frequently used as a redox-active substance in redox cycling sensor arrangements. An oxidation potential at approximately 260 mV and a reduction potential at approximately 10 mV with regard to a silver-silver chloride reference electrode is shown in diagram 200. As is visible from the cyclic voltammogram of FIG. 2, a significant rise or fall of the electrical current ensues at very high or very low electrical voltages, which is ascribed to additional unwanted electrochemical reactions of additional components in an electrolyte.

For the functionality of a redox cycling sensor arrangement, it is thus important to correctly adjust the electrical potentials to the electrodes with regard to the electrical potential of the electrolyte.

An interdigital electrode arrangement 300 that comprises two finger-shaped interlocking electrodes (namely a generator electrode 301 and a collector electrode 302) known from the prior art is shown in FIG. 3. A reference electrode 303 and a counter-electrode 304 are also shown. The electrodes 301 through 304 are formed on a substrate 305. An electrolytic analyte (not shown) that is coupled with the electrodes 301 through 304 can be applied on the interdigital electrode arrangement 300. The electrical potential of the electrolytic analyte is provided to an inverted input of a comparator 306 by way of the reference electrode 306, and is compared by this with a desired electrical potential at the non-inverted input of the comparator 306. Given a deviation of the electrical potential of the reference electrode 303 from the desired potential, the counter-electrode 304 is modulated via an output of the comparator 306 such that this subsequently delivers electrical charge carriers as needed in order to maintain the desired electrical potential of the electrolyte. Together with the comparator 306 and the counter-electrode 304, the reference electrical 303 clearly forms a potentiostat device. The electrical potentials at the operating electrodes 301, 302 are adjusted relative to the reference voltage. Electrical sensor currents of the generator electrode 301 or of the collector electrode 302 which contain information about a possible occurred sensor event are detected by way of first and second amp meters 307, 308.

What is important in the operation of the interdigital electrode arrangement 300 is the correct detection of the electrical potential of the electrolytic analyte, i.e., a sufficiently good and reliably-functioning reference electrode 303. This, in particular, frequently represents a large problem in sensor arrangements realized by way of an integrated circuit. For example, in order to monolithically integrate a silver-silver chloride reference electrode, a plurality of additional process steps are necessary, which represents a significant cost expenditure.

A sensor arrangement 400 according to the prior art is shown in FIG. 4, in which the interrelationships described with reference to FIG. 3 are shown more precisely.

As shown in FIG. 4, the generator electrode 301, the collector electrode 302, the reference electrode 303 and the counter-electrode 304 together with a possibly filled analyte form the electrochemical system 401. The four electrodes 301 through 304 are electrochemically, electrically coupled by way of the electrolytic analyte (not shown). A potentiostat is formed from the reference electrode 303 (which measures the electrochemical potential of the electrolytic analyte) and a first operation amplifier 402 (or control amplifier) that readjusts the electrical potential of the analyte at its output via the counter-electrode 304. The desired value for the potential to be adjusted of the electrolytic analyte is designated as “AGND” (“Analog Ground”).

For a correct functioning of the circuit, AGND has to lie between the positive and the negative operating voltage of the circuit. The value of AGND typically lies in the middle between the two operating voltages. However, the absolute value of AGND is not decisive for the functioning of the sensor arrangement 400, since only the potential difference between the electrodes 301, 302 is relevant for the electrochemical system 401. The electrical voltages at the sensor electrodes 301, 302 are adjusted relative to the reference voltage AGND using the second and third operation amplifier 403, 404 (or control amplifier). The generator electrode 301 is brought to AGND+V_ox and the collector electrode 302 is brought to AGND+V_red, whereby V_ox is the oxidation potential and V_red is the reduction potential of the redox-active substance (for para-aminophenol, V_ox=260 mV, V_red=10 mV; see FIG. 2).

Electrical sensor currents at the electrodes 301, 302 are reflected and amplified via a first and via a third p-MOS field effect transistor for the generator electrode 301 or via a first and a third n-MOS field effect transistor 408, 410 for the collector electrode 302. A second n-MOS field effect transistor 406 and a second p-MOS field effect transistor 407 are coupled with outputs of the respective operation amplifier 403, 404. A sensor event on the sensor signal characterizing the generator electrode 301 is provided at a first sensor current output 411. A sensor event on the sensor signal characterizing the collector electrode 302 is provided at a second sensor current output 412. The sensor events are correlated on both electrodes.

The circuit of the sensor arrangement 400 is imperatively based on a correct detection of the electrical potential of the analyte using the reference electrode 303.

Possibilities known from the prior art for realization of such a reference electrode and physical properties of such a reference electrode are described in the following.

In electrochemical analyses, ingredients of an analyte are determined based on a variation of the electrical potential at operating electrodes or by way of detection of an electrical current flow at operating electrodes. According to the Nernst equation, a redox system as it is used in many biosensors for detection of sensor events exhibits a characteristic potential at which oxidations or reductions can ensue. This potential depends on the concentration ratios and on the temperature. The Nernst equation reads:
E=E₀+RT/(nF)log([Ox]/[Red]) (1)

In equation (1), E is the electrical potential and E₀ is a reference potential, for example, a potential under standard conditions. R is the gas constant, T is the absolute temperature, n is an electrochemical valence and F is the Faraday constant. A concentration of an oxidized species is designated as [Ox]; a concentration of a reduced species is designated as [Red].

As is clear from (1), the electrical potential E represents no absolute value, but rather is relative to a reference potential E₀. A normal hydrogen electrode is typically used as a reference electrode, and all electrochemical voltages are relative to the potential of such a reference electrode. Instead of such reference electrodes (what are known as reference electrodes of the first type) that require a high industrial cost, in electrochemistry, reference electrodes of the second type are typically used such as a silver-silver chloride reference electrode or a calomel electrode.

A silver-silver chloride reference electrode not integrated on the chip can be used for sensor arrangements based on integrated circuits. However, such a reference electrode is very expensive and, as a separate component, runs counter to an intended integration and pursued miniaturization. The integration of a silver-silver chloride reference electrode is technically difficult and requires a significant effort.

What are known as quasi-reference electrodes or pseudo-reference electrodes are also used in microchip-based analysis systems. Such electrodes are comprised of a noble metal (for example, gold) that is in contact with the electrolyte. Since such noble metal electrodes are essentially inert, their electrochemical potential is essentially constant. However, if the chemical composition of an analyte changes during an experiment, the reference voltage of such a quasi-reference electrode can shift. This shift can be so large that an error-free operation of the electrochemical analysis system is no longer assured.

Given a shift of the electrical potential, the measured electrochemical signals are no longer meaningful since it is no longer well defined which substance is converted on the operating electrode. Given very significant shifts, the electrode material itself can even be electrochemically oxidized and dissolve into the solution (for example, given gold electrodes, as AuCl₃). In this case, the analysis system is irreversibly destroyed.

Quasi-reference electrodes exhibit the problem that the measured electrical potential is not independent of an analyte and can thus drift in the course of the measurement period. An increasingly false potential of the analyte can thereby be displayed. If this drift is too large, the redox reactions can come to a standstill, since one of the two redox potentials is no longer achieved. Given even larger deviations, additional reactions can run at the electrodes; a region of significantly rising or falling flanks is achieved (see the curve in FIG. 2). A distinct material flow between one of the two operating electrodes and the counter-electrode hereby occurs. If this state continues for a long time, the electrodes are, for example, destroyed due to electro-migration, or this state leads to gas formation at the electrodes via electrolysis.

International Patent Publication No. WO 87/03095 discloses a capacitive sensor for a chemical analysis and measurement in which the concentration of an analyte in a fluid is determined using a biochemical bond system.

German Patent Document No. DE 196 10 115 A1 discloses a method for detection of molecules or molecule complexes, whereby a test sample is brought into contact with an ultra-microelectrode arrangement which comprises at least two electrode structures.

German Patent Document No. DE 199 16 921 A1 discloses an electrical sensor array based on voltametric and/or impedimetric detection principles for application in analytical biochemistry, diagnostics and environmental monitoring.

U.S. Pat. No. 4,822,566 discloses a device for detection for the presence and/or to measure the concentration of an analyte in a fluid medium.

### SUMMARY

The invention is in particular based on the problem to provide a sensor arrangement in which the problems of a reference electrode known from the prior art are avoided.

The problem is solved via a sensor arrangement for detecting particles that may be contained in an electrolytic analyte, comprising: an operating electrode that can be electrically coupled with the electrolytic analyte, the operating electrode being arranged such that sensor events occur at the operating electrode given a presence in the sensor arrangement of the electrolytic analyte comprising possible particles to be detected; an additional electrode that can be electrically coupled with the electrolytic analyte; an operating circuit coupled with the operating electrode, which operating circuit is arranged such that it adjusts an essentially constant potential difference between the operating electrode and the additional electrode; and a device that is arranged such that it keeps essentially constant a ratio of electrical currents flowing at the operating electrode and the additional electrode. The problem is also solved with a sensor array comprising a plurality of sensor arrangements as described above.

Various embodiment of the inventive sensor arrangement for detection of particles possibly contained in an electrolytic analyte comprise an operating electrode that can be electrically coupled with the electrolytic analyte, which operating electrode is arranged such that sensor events occur in the sensor arrangement at the operating electrode given the presence of the electrolytic analyte comprising the possible particles to be detected.

An additional electrode that can be electrically coupled with the electrolytic analyte is also provided in the sensor arrangement. Moreover, the sensor arrangement comprises an operating circuit coupled with the operating electrode, which operating circuit is arranged such that it adjusts an essentially constant potential difference between the operating electrode and the additional electrode. The sensor arrangement also comprises a device that is arranged such that it essentially holds constant a ratio of electrical currents flowing at the operating electrode and the additional electrode.

These embodiments of the inventive sensor array comprise a plurality of sensor arrangements with the features described above.

A fundamental idea is to set constant the electrical voltage between two electrodes of the sensor arrangement (which can particularly be realized as a two-electrode sensor arrangement) and to keep constant the ratio of electrical currents at both electrodes (for example, currents of the same magnitude and different polarity sign). Based on this measure, a reference electrode is entirely avoided, such that the significant effort required according to the prior art to fashion such a reference electrode is saved. Expressed otherwise, the sensor arrangement is free of a reference electrode, since the effect of a reference electrode according to the prior art is replaced by the effect of the operating circuit and the device for maintaining the ratio of the electrical currents flowing at the operating electrode and the additional electrode. In spite of economizing on a reference electrode, in the present sensor arrangement, a more stable operation of the sensor arrangement (which, for example, can be realized as an electrochemical sensor arrangement) is ensured.

Instead of the detection of the electrochemical potential of an analyte using a reference electrode, as required according to the prior art, a potential difference between two electrodes of the sensor arrangement (for example, between two operating electrodes of a redox cycling sensor arrangement) is held constant and the ratio of the electrical currents at both electrodes is likewise held constant.

Given large currents of equal magnitude, a two-electrode sensor arrangement is clearly enabled in contrast to a four-electrode arrangement according to the prior art, with operating electrodes, reference electrode and counter-electrode.

It is a significant advantage of various embodiments of the inventive sensor arrangement or sensor array that a reference electrode as it is required according to the prior art is dispensable. In particular, in highly-developed miniaturized systems, the integration of a reference electrode is linked with extraordinarily large process-related (and thus financial) expenditure. In spite of the economization on the reference electrode, embodiments of the inventive arrangement enables a higher operational reliability than given an operation with quasi-reference electrodes used according to the prior art, which merely supply concentration-dependent potentials.

In the following, different types of electrodes are characterized as they are used in sensor arrangements.

An operating electrode is to be understood as an electrode that is coupled with an electrolytic analyte and at which the (in particular electrochemical) reactions ensue that are relevant for a sensor event. Examples for operating electrodes are generator and collector electrodes of a redox cycling sensor arrangement.

A counter-electrode is to be understood as an electrode that is coupled with an electrolytic analyte, and this provides electrical charge carriers as needed in order to adjust a predetermined electrochemical potential of the analyte.

A reference electrode (that is avoided according to various embodiments of the invention described herein) is to be understood as an electrode that is coupled with an electrolytic analyte and whose electrochemical potential is determined or sensed.

Preferred developments of the inventions result from the various embodiments of the invention discussed herein.

In various embodiments of the inventive sensor arrangement, the electrolytic analyte can comprise a substance bound to the particles to be detected, with a first redox potential in a first concentration, and a secondary substance with a second redox potential in a second concentration in the electrolytic analyte, whereby the second concentration is preferably at least as high as the first concentration. According to the described development, given sensor events at the operating electrode, an electrochemical reaction also ensues with the participation of the substance bound to the particles to be detected.

The oxidation or reduction potential of the known secondary substance (preferably present in surplus) can be demonstratively used as a reference point, which secondary substance can be provided in the analyte or as an immobilized layer on one of the two electrodes (for example, in an embodiment as a counter-electrode). The core of the described development is thus to replace the reference electrode required according to the prior art by way of the addition of the substance and of the secondary substance in the analyte as well as by way of the described circuit technology for operation of the electrochemical analysis system. A more reliable and stable operation of the electrochemical, miniaturized analysis system is thereby also ensured without a reference electrode.

The sensor arrangement can also be arranged such that the essentially constant potential difference between the operating electrode and the additional electrode is set to a value that is equal to or larger than the difference between the first redox potential and the second redox potential. In other words, the knowledge of the first redox potential of substance bound to the particles to be detected and the knowledge of the second redox potential of the secondary substance are preferably used in surplus concentration in order to adjust a suitable value of the potential difference between both electrodes of the sensor arrangement (for example, in the case of para-aminophenol to approximately 250 mV or more, (compare FIG. 2)).

The operating circuit can also be arranged such that, in the case of sensor events, it provides an electrical sensor signal characterizing the sensor events. This sensor signal can, for example, be a sensor current or a sensor voltage. The sensor signal can also be pre-processed on-chip, for example, digitized and/or amplified in order to improve the signal-to-noise ratio.

The sensor arrangement of the invention can be monolithically integrated into and/or on a substrate. The substrate can, for example, be a semiconductor substrate (such as a silicon wafer or a silicon chip). The sensor arrangement can thereby be formed as a miniaturized integrated circuit. The sub-circuit or sub-circuits of the sensor arrangement (for example, the operating circuit) can, for example, be provided with the electrodes below the electrochemical system, which enables a particularly space-saving configuration.

Alternatively, at least one first part of the components of the sensor arrangement can be provided external from (i.e., separate from) a substrate in and/or on which is fashioned a second part of the components of the sensor arrangement.

The sensor arrangement can be arranged as an electrochemical sensor arrangement for detection of oxidizable or reducible substances.

The sensor arrangement can be arranged as a biosensor arrangement for detection of biomolecules, particularly macromolecular biopolymers (for example, DNA half-strands, proteins, enzymes, polymers, and oligomers).

The sensor arrangement can be arranged for detection of DNA molecules, oligonucleotides, polypeptides and/or proteins.

In the sensor arrangement, capture molecules can be immobilized at least on the operating electrode.

The sensor arrangement can particularly be arranged as a redox cycling sensor arrangement, i.e., as a sensor arrangement in which the method described with registration to FIG. 1A, FIG. 1B can be implemented, however, without a reference electrode being required for this.

The sensor arrangement can also be arranged as a dynamic biosensor arrangement. A “dynamic” biosensor arrangement is particularly understood as such a biosensor arrangement that is not only operating quasi-statically, but rather in which dynamic (meaning significantly variable over time) measurement signals (for example, voltage spikes and alternating voltage voltammetry) occur.

The operating electrode and the additional electrode can exhibit an essentially equally-large area. For a stable operation of the sensor arrangement, it is advantageous that the surface of the additional electrode (for example, a counter-electrode) approximately equally corresponds to the surface of the operating electrode. In this case, the current density on both electrodes can be kept sufficiently low in order to prevent unwanted reactions. In the electrochemistry according to the prior art, the rule of thumb is typically used that the counter-electrode approximately exhibits ten to one hundred times the surface of the operating electrode. This is necessary according to the prior art since a potential shift of the electrolyte the significantly ensues on the capacitive coupling of the counter electrode with the electrolyte via double-layer capacitance, and only negligible reactions occur on the counter-electrode. In this case, the voltage rise that a potentiostat causes at the counter-electrode has a negligibly small amplitude.

Particularly given the embodiment of the inventive sensor arrangement in which a substance to be reduced is added in a sufficiently high (surplus) concentration, the surface of the counter-electrode can be significantly smaller than according to the prior art and lie in the range of the surface of the operating electrode or be realized approximately equal in surface with the operating electrode. Particularly in miniaturized electrochemical analysis systems, this represents a real advantage, such that the integration density can be increased.

In the sensor arrangement according to various embodiments of the invention, the device that is arranged such that it keeps essentially constant a ratio of an electrical current flowing on the operating electrode and the additional electrode can be a circuit, meaning that it can be realized according to circuit technology.

This development is based on the realization that, for a correction functionality of the sensor arrangement (for example, according to the redox cycling method on the one hand) the voltage difference between the two electrodes (that, according to the redox cycling method, are both operating electrodes) should correspond to at least the difference between oxidation and redox potential. On the other hand, the current flow at the two operating electrodes should be equal in terms of magnitude or at least stand at a fixed (i.e., constant) ratio relative to one another. Thus according to various embodiments of the invention, the electrical potentials at all electrodes are not necessarily set; rather, only the voltage difference between collector electrode and generator electrode is to be set. According to the described development, a predeterminable ratio between the currents at both sensor electrodes is now enforced using circuited measures. The correct voltage values then adjust themselves via the electrolytic analyte.

The additional electrode can be an additional operating electrode that is arranged such that sensor events occur at the additional operating electrode given the presence in the sensor arrangement of an electrolytic analyte comprising possible particles to be detected. In other words, both operating electrodes and additional electrodes can be fashioned as operating electrodes. This development is, for example, advantageous in a redox cycling sensor arrangement in which the operating electrode and the additional electrode of the inventive sensor arrangement are realized as a collector and generator electrode.

According to the described development, the operating circuit can also comprise a counter-electrode that can be electrically coupled with an electrolytic analyte, which counter-electrode is arranged such that electrical charge carriers are provided to the electrolyte as needed by way of the counter-electrode based on a comparison of the electrical currents at the operating electrode and at the additional operating electrode, such that essentially a constant potential difference is set between the operating electrode and the additional operating electrode. According to this embodiment, the inventive sensor arrangement is a three-electrode sensor arrangement with two operating electrodes and a counter electrode. The electrical currents at both operating electrodes are compared with one another, for example, subtracted from one another, electrical charge carriers of suitable amount and polarity sign are additionally supplied to the electrolytic analyte via the counter-electrode based on this comparison value, such that a constant potential difference (or a constant ratio of the currents at both operating electrodes) is enabled.

The electrical circuit can also comprise a current reflector circuit which is connected such that it essentially provides the amount of the electrical current strength at the operating electrode to the additional operating electrode. In other words, given negligibly small inaccuracies of a current reflector circuit, a constant amount between the currents at operating electrode and additional operating electrode can be ensured, in that an electrical current at the operating electrode is coupled into the additional operating electrode using the current reflector with a copy factor of (in an ideal manner) “minus one”.

The operating circuit of the sensor arrangement can comprise a source follower and precisely one operation amplifier. Given the realization of an operating circuit in the case of two operating electrodes, two operation amplifiers are frequently necessary when sensor signals should be provided in the operating circuit. Using a source follower, one of the two operation amplifiers can be economized on, which has a reduced circuit expenditure and a space savings as a result.

As an alternative to a circuited realization, the setup of the sensor arrangement can be executed as an insulation device that is arranged such that it electrically insulates the electrolytic analyte electrically coupled with the operating electrode and the additional electrode from the environment of the electrolytic analyte. Expressed differently, due to Kirchoffs laws, a fixed relationship arises between the currents at both electrodes given a sensor arrangement in which (only) the operating electrode and the additional electrode are coupled with an electrolytic analyte (i.e., are immersed in this). The described development inasmuch represents a mechanical realization of the arrangement.

The additional electrode can be a constant potential electrode that is brought to a constant electrical potential. The additional electrode is thus not necessarily coupled with the operating circuit, but rather can also be brought to a constant electrical potential (for example, the electrical ground potential).

In the sensor arrangement, the operating electrode can also be provided with a functionalization (for example, capture molecule with which particles to be detected can hybridize), at which functionalization sensor events can occur, and the additional electrode can be provided with charge carrier reservoir material that, in the case of sensor events at the operating electrode, provides electrical charge carriers for buffering of current surges due to sensor events at the operating electrode. In a dynamic system in which very many oxidation or reduction events can ensue at the operating electrode in a very short time, it is advantageous that a sufficient quantity of convertible materials that can additionally supply charge carriers are immobilized at the additional electrode, for example, in its execution as a counter-electrode. The time constant of the system can thereby be kept low, and an exact control of the electrode potentials is enabled.

The sensor arrangement of the invention can also comprise a constant potential electrode electrically coupled with the electrolyte, which constant potential electrode is brought to a constant electrical potential. Such an additional electrode, which can, for example, be brought to the electrical ground potential, can provide a constant electrical potential to the electrolytic analyte.

In the following, the sensor array that comprises the previously discussed sensor arrangement is described in detail. Embodiments of the sensor array also apply for the sensor arrangement and vice versa.

The sensor arrangements can primarily be arranged matrix-like in the inventive sensor array. This enables a particularly high integration density of the sensor arrangements, which is particularly advantageous for high-throughput analyses in which each sensor arrangement is sensitive to a different biomolecule, for example, to oligonucleotides of different base sequences.

The sensor array can comprise a control circuit that is arranged for activation, selection and/or readout of a sensor arrangement or of a part of the sensor arrangement (for example, a row or column of sensor arrangements). Such a control circuit, which can be integrated provided on and/or in a chip or can be provided external from the chip, frequently comprises a plurality of selection transistors, and row and column conductors, in order to specifically activate and select individual sensor arrangements or read out a sensor signal.

The additional electrodes can be provided in common for at least one part of the sensor arrangements of the sensor array and can be arranged as a constant potential electrode that is brought to a constant electrical potential (for example, ground potential). A particularly space-saving arrangement can be mutually achieved for one part or all of the sensor arrangements by providing the additional electrode.

In the sensor array, at least in a part of the sensor arrangement the respective additional electrode can also be coupled with the respective operating circuit, and a common constant potential electrode can be provided that is brought to a constant electrical potential. This embodiment also enables an advantageous and space-saving arrangement of the sensor arrangements of the sensor array.

### DESCRIPTION OF THE DRAWINGS

Exemplary embodiments of the invention are shown in Figures and are explained in detail in the following. Identical or similar components in different Figures are provided with the same reference characters. The representations in the Figures are schematic and not to scale.

FIGS. 1A, 1B is a schematic diagram illustrating different operating states of a redox cycling sensor arrangement according to the prior art;

FIG. 2 is a schematic diagram which shows a known cyclic voltammogram for para-aminophenol;

FIG. 3 is a circuit pictorial schematic of an interdigital electrode arrangement according to the prior art;

FIG. 4 is a circuit schematic of a sensor arrangement according to the prior art;

FIG. 5 is a graph that shows a current potential characteristic according to the prior art;

FIG. 6 is a graph that shows a current potential characteristic according to an exemplary embodiment of the invention;

FIG. 7 is a circuit schematic showing a sensor arrangement according to a first exemplary embodiment of the invention;

FIG. 8 is a circuit schematic showing a sensor arrangement according to a second exemplary embodiment of the invention;

FIG. 9 is a circuit schematic showing a sensor arrangement according to a third exemplary embodiment of the invention;

FIG. 10 is a circuit schematic showing a sensor array according to a first exemplary embodiment of the invention;

FIG. 11 is a circuit schematic showing a sensor array according to a second exemplary embodiment of the invention;

FIG. 12 is a circuit schematic showing a sensor arrangement according to a fourth exemplary embodiment of the invention;

FIG. 13 is a circuit schematic showing a sensor arrangement according to a fifth exemplary embodiment of the invention;

FIG. 14 is a circuit schematic showing a sensor arrangement according to a sixth exemplary embodiment of the invention;

FIG. 15 is a circuit schematic showing a sensor arrangement according to a seventh exemplary embodiment of the invention; and

FIG. 16 is a circuit schematic showing a sensor arrangement according to a eighth exemplary embodiment of the invention.

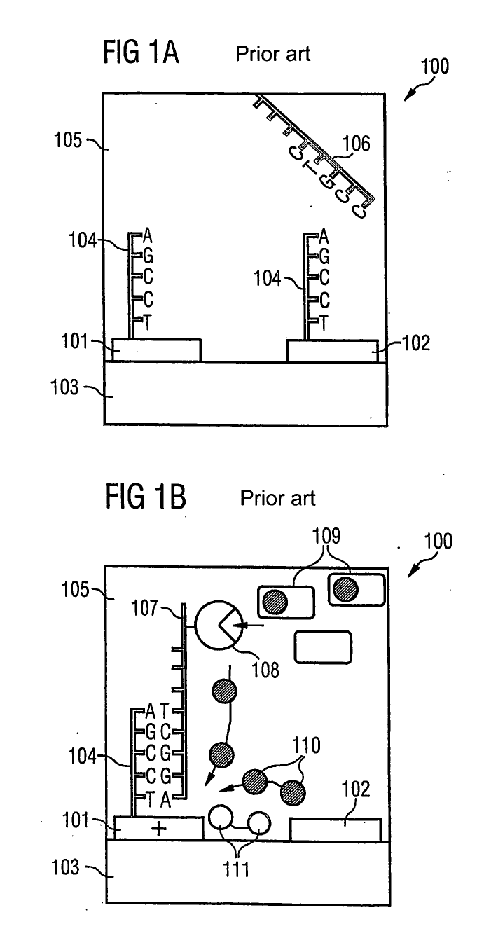

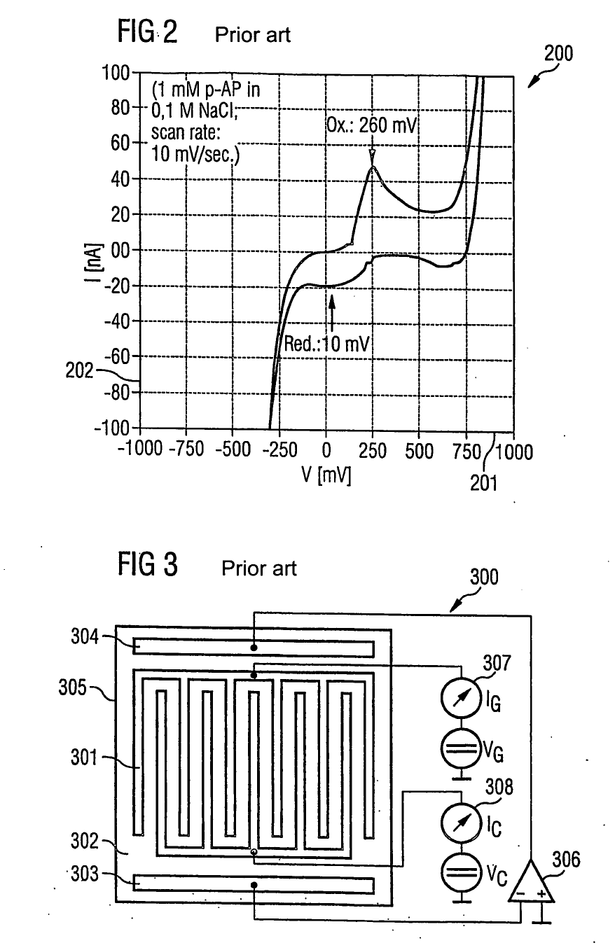

### DETAILED DESCRIPTION OF THE PREFERRED EMBODIMENTS

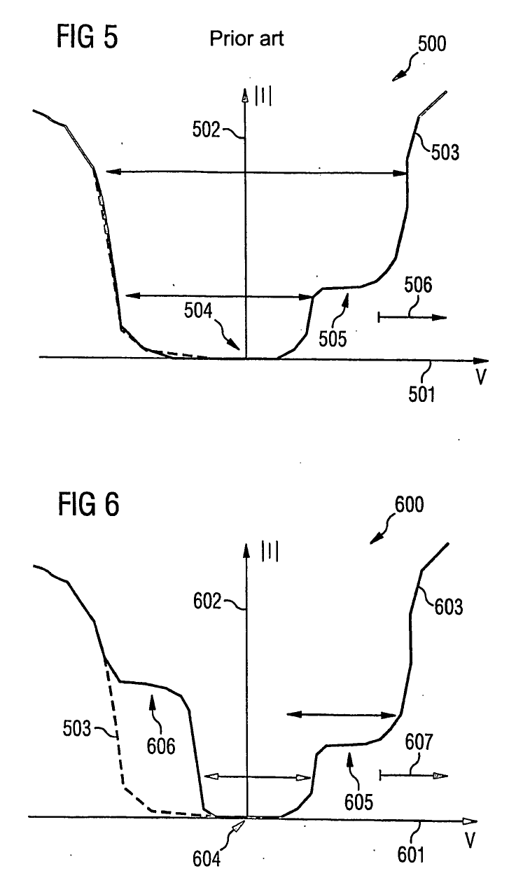

In the following, a significant aspect of the invention is described with reference to FIG. 5 and FIG. 6 in delimitation from the prior art. For this, electrode current magnitude-electrode voltage curves for a sensor arrangement according to the prior art are explained with reference to FIG. 5 and according to various embodiments of the invention with reference to FIG. 6.

In FIG. 5 a diagram 500 is shown along whose abscissa 501 an electrode voltage is plotted, contrary to which along whose ordinate 502 an electrode current in terms of magnitude is plotted for a redox sensor arrangement according to the prior art.

The range of positive electrode voltages in FIG. 5 graphically corresponds to the range in which oxidations occur at an electrode, in contrast to which reductions ensue at an electrode in the range of the electrode voltage with negative polarity sign. In FIG. 5, a curve is exemplarily shown that shows an electrode current magnitude-electrode voltage curve 503 at a sensor arrangement (known from the prior art) with a reference electrode. The coordinate origin of the diagram 500 corresponds to a rest point 504 or rest potential of the sensor electrode. At a range 505, unwanted reactions ensue at one of the operating electrodes with the involvement of particles to be detected, in contrast to which unwanted reactions of components additionally contained in an electrolytic analyte ensue in a range 506.

A schematic representation of the magnitude of the electrical current at an electrode in an electrochemical system according to the prior art when its electrical potential is increased or decreased starting from the rest point 504 is graphically shown in FIG. 5. Given an increase of the electrical potential at an operating electrode, the desired oxidations initially start; see range 505. These oxidations can, for example, be oxidations of ferrocene markers at DNA half-strands to be detected. Given a further increase of the electrical voltage, additional substances of the electrochemical system are converted in an unwanted manner; see range 506. A counter-electrode compensates the current flowing at the operating electrode and for this is brought to a negative electrical potential by a potentiostat circuit. According to the prior art, this potential is not precisely determined, but rather depends on the content substances of the electrolyte. According to the prior art, the potential at the operating electrode is determined using a reference electrode that determines the potential of the electrolyte independent of concentration. The operating electrode potential is then adjusted relative to this electrical potential.

One aspect of the invention is explained using the diagram 600 shown in FIG. 6, along whose abscissa 601 an electrode voltage is plotted, in contrast to which along whose ordinate 602 the electrode current in terms of magnitude is plotted.

An electrode current-electrode voltage curve 603 is also shown in FIG. 6, which curve, however, differs from the electrode current magnitude-electrode voltage curve 503 from FIG. 5 in a range of negative electrode voltages. The coordinate origin in FIG. 6 likewise corresponds to a rest point 604 or a rest potential. A range of first desired reactions 605 and a range of second desired reactions 606 as well as a range of unwanted reactions 607 are also shown in FIG. 6.

The current (in terms of magnitude) in an electrode in an embodiment of the inventive electrochemical sensor arrangement when the potential of the electrode is increased or decreased from the rest point 604 is graphically shown in FIG. 6. Given an increase of the electrode potential at the operating electrode (i.e., in the range of positive electrode voltages on the abscissa 601), the desired oxidations initially start; see range 605. These oxidations can, for example, be oxidations of ferrocene markers on DNA half-strands.

Given even higher electrical voltages, additional substances of the electrochemical system are converted, which is not desirable; see range 607. A counter-electrode has to compensate the current flowing at the operating electrode and is brought to a sufficiently negative potential for this, for example, by a potentiostat circuit. In contrast to the prior art, where this potential is not precisely determined and depends on the content substances of the analyte, in an embodiment of the inventive sensor arrangement, a secondary substance of sufficiently high concentration is added to the electrolyte, the reduction potential of which secondary substance lies comparably close to the rest potential 604 of the counter-electrode, at least closer than the reduction potential of other content substances of the electrolytes (see FIG. 6, range of negative voltages). If the counter-electrode is now brought to a negative electrical potential by the potentiostat circuit, this can already apply the necessary current given a slight deflection of the potential from the rest position 604, since the secondary substance added in surplus is initially reduced in the electrolyte.

It is hereby utilized that both the oxidation potential of the substance to react at the operating electrode and the reduction potential of the added secondary substance are known. The knowledge of both potentials relative to a reference potential is not crucial for a successful operation of the analysis system; rather, the knowledge of the difference of both potentials is sufficient. This difference is independent of a reference potential and shows only a weak deviation from the composition of the electrolyte.

### Sensor Arrangement—First Embodiment

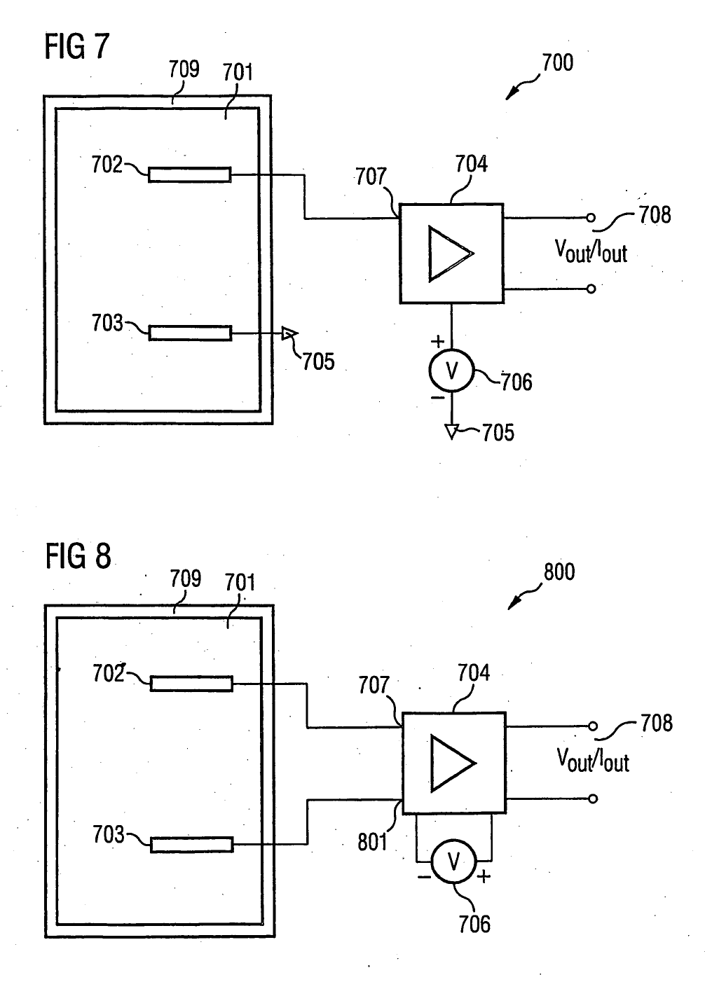

In the following, a sensor arrangement 700 according to a first exemplary embodiment of the invention is described with reference to FIG. 7.

In the sensor arrangement 700, an electrolytic analyte 701 is provided in a reaction volume and electrically insulated from the environment by way of an insulation device 709. An operating electrode 702 and a counter-electrode 703 are immersed in the electrolytic analyte. The sensor arrangement 700 also comprises an operating circuit 704 with an input 707 which is coupled with the operating electrode 702. The counter-electrode is brought to the electrical ground potential 705. The operating circuit 704 is also arranged such that it can provide a positive potential of an electrode potential device 706 to the operating electrode 702 such that a constant voltage is applied between the operating electrode 702 and the counter electrode 703 located at the ground potential. A measurement value (meaning a sensor signal), for example, a voltage Vₒᵤₜ or a current Iₒᵤₜ is provided at an output 708 of the operating circuit 704.

The sensor arrangement 700 is arranged for detection of particles possibly contained in the electrolytic analyte 701. The operating electrode 702 is arranged such that sensor events occur in the sensor arrangement 700 at the operating electrode 702 in the presence of the analyte 701 comprising possible particles to be detected. For this, capture molecules that can hybridize with DNA half-strands possibly contained in the analyte 701 are immobilized at the operating electrode 702. The counter-electrode 703 is electrically coupled with the electrolytic analyte 701 in that it is immersed in the analyte 701. The operating circuit 704 is arranged such that it adjusts a constant potential difference B between the electrodes 702, 703. The insulation device 709 allows that the electrical currents flowing at the operating electrode 702 and at the counter-electrode 703 are the same in terms of magnitude.

The sensor arrangement 700 is thus arranged as a two-electrode system in which both the operating electrode 702 and the counter-electrode 703 are immersed in the same reaction volume (analyte 701) which is electrically insulated from the environment by way of the insulation device 709. The electrolyte potential is consequently adjusted to a value at which the same current (in terms of magnitude) flows at both electrodes 702, 703 (Kirchhoff laws). The potential difference between the two electrodes 702, 703 is controlled by way of the operating circuit 704 and electrode potential device 706 and adjusted to a value that is suitable for operation of the sensor arrangement 700.

The sensor arrangement 700 is well-suited for a quasi-static sensor system such as a sensor arrangement based on the redox cycling principle. In a redox cycling sensor, a secondary substance with sufficiently high concentration is already inherently present and does not have to be additionally added. In redox cycling (for example, using para-aminophenol), the redox-active substance is a substance with known redox behavior. An electrode current magnitude-electrode voltage curve as it is, for example, shown in FIG. 6 can be obtained with the sensor arrangement 700.

### Sensor Arrangement—Second Embodiment

In the following, a sensor arrangement 800 according to a second exemplary embodiment of the invention is described with reference to FIG. 8.

The sensor arrangement 800 shown in FIG. 8 essentially differs from the sensor arrangement 700 shown in FIG. 7 in that the counter-electrode 703 is not located at the electrical ground potential 705, but rather is coupled with the operating circuit 704 via an additional input 801 of the operating circuit 704. A predetermined voltage V is thus applied between the electrodes 702, 703 by way of the operating circuit 704 using the electrode potential device 706.

A difference between the sensor arrangement 700 and the sensor arrangement 800 shown in FIG. 8 is that the electrical potential of the counter-electrode 703 is predeterminately fixed in the sensor arrangement, in that the counter-electrode 703 in FIG. 7 is brought to the electrical ground potential 705. In contrast to this, in the sensor arrangement 800, the electrical potential of both electrodes 702, 703 is free. In the sensor arrangement 800, the operating circuit 704 ensures that the electrical potential of both electrodes 702, 703 does not leave a usable voltage range of the operating circuit 704.

### Sensor Arrangement—Third Embodiment

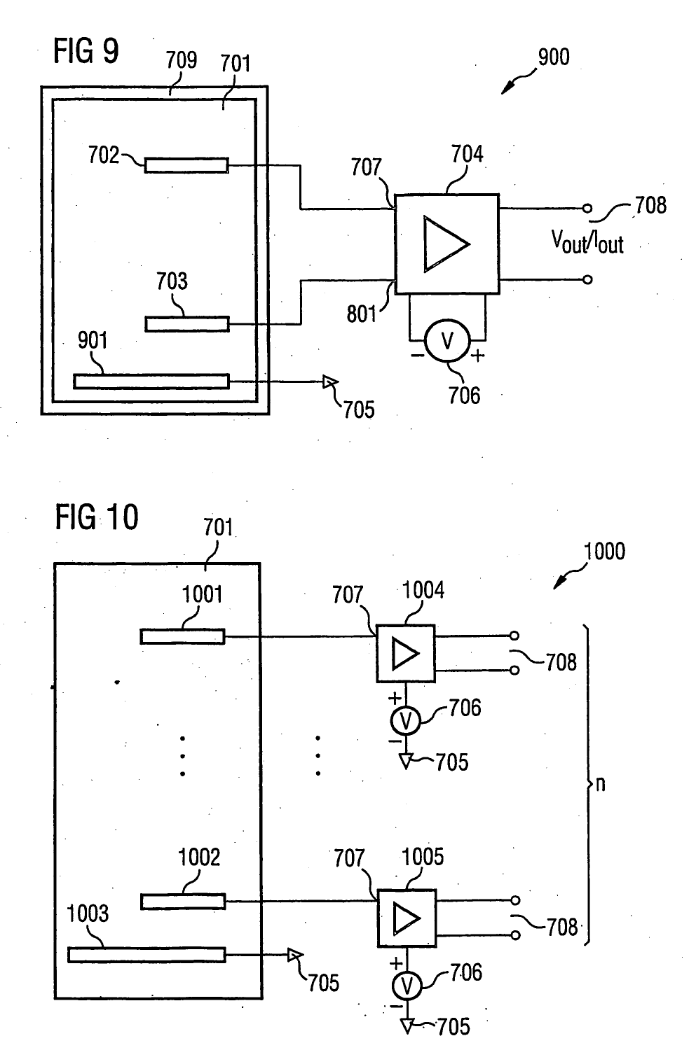

In the following, a sensor arrangement 900 according to a third exemplary embodiment of the invention is described with reference to FIG. 9.

The sensor arrangement 900 essentially differs from the sensor arrangement 800 shown in FIG. 8 in that a ground electrode 901 is provided in the sensor arrangement 900 in addition to the other components of the sensor arrangement 800. The ground electrode 901 is immersed in the electrolytic analyte 701 and thus coupled with the analyte 701. An insulation device 709 for delimitation of the reaction volume is also provided in the sensor arrangement 900. An additional difference between the sensor arrangement 900 and the sensor arrangement 800 is to be seen in that, due to the addition of the ground electrode 901, the electrical currents at the electrodes 702, 703 henceforth do not unavoidably have to be equally large in terms of magnitude. Due to the application of an externally predetermined potential (namely the electrical ground potential 705) at the electrolytic analyte 701, the Kirchhoff laws now no longer necessarily lead to the electrical currents at the electrodes 702, 703 being equal in terms of magnitude.

Nevertheless, in order to ensure that a ratio of the electrical currents at the operating electrode 702 and at the counter-electrode 703 is essentially constant, in the sensor arrangement 900 the operating circuit 704 is arranged such that the ratio of both of these currents remains essentially constant. In other words, a constant current ratio is enabled in the sensor arrangement 900 due to a circuited configuration. The operating circuit 704 can behave like an ideal current source at the electrode connections 707, 801. The potential of the electrolytic analyte 701 is established by way of the additional ground electrode 901.

### Sensor Array—First Embodiment

In the following, a sensor array 1000 according to a first exemplary embodiment of the invention is described with reference to FIG. 10.

The sensor array 1000 comprises a reaction volume into which an electrolytic analyte 701 is filled. A first operating electrode 1001, and a second operating electrode (not shown) through an n-th operating electrode 1002 are immersed in the electrolytic analyte 701. A counter-electrode 1003 common for all sensor arrangements of the sensor array 1000 is also immersed in the analyte, which common counter-electrode 1003 is brought to an electrical ground potential 705. An operating circuit 1004, 1005 is associated with each of the first through n-th operating electrodes 1001,1002.

A first operating circuit 1004 is associated with the first operating electrode 1001, a second operating circuit (not shown) is associated with the second operating electrode, and so on, with an n-th operating circuit 1005 is associated with the n-th operating electrode 1002. Each operating electrode is coupled with an input 707 of the associated operating circuit. A sensor signal in the form of a sensor current, a sensor voltage, etc., which is characteristic for sensor events that have occurred at the associated operating electrode, is provided at a respective output 708 of a respective operating circuit. Each operating circuit comprises an electrode potential device 706 which is arranged such that it keeps constant the electrical potential between the operating electrode associated with the respective operating circuit and the common counter-electrode 1003.

In the sensor array 1000, in total, n units of operating electrodes and associated operating circuits are thus provided, which n units or sensor arrangements can, for example, be arranged in a matrix shape (which is not shown in the schematic representation of FIG. 10). Also not shown in FIG. 10 are selection and control electronics for selection, control or readout from one of the respective operating electrodes. The sensor array 1000 is graphically an arrangement of a plurality of sensor arrangements that are connected as a high-integrity analysis system and can be operated in common. The common counter-electrode 1003 can be provided with a surface that is significantly larger than each individual surface of one of the operating electrodes 1001,1002.

### Sensor Array—Second Embodiment

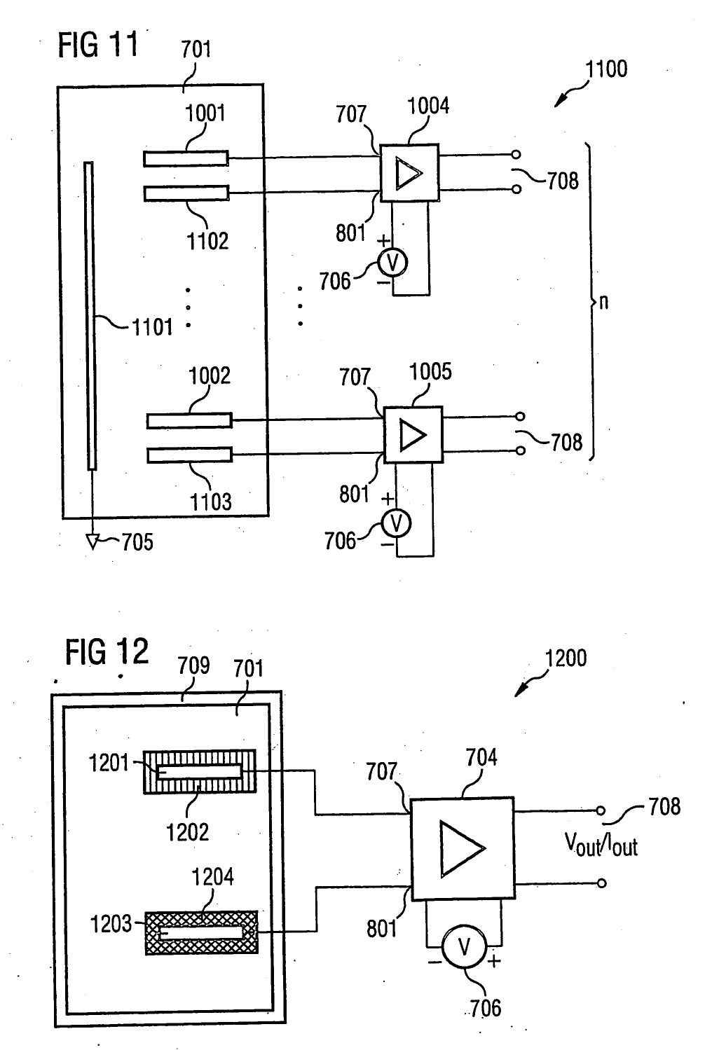

In the following, a sensor array 1100 according to a second exemplary embodiment of the invention is described with reference to FIG. 11.

The sensor array 1100 essentially differs from the sensor array 1000 shown in FIG. 10 in that an individual counter-electrode 1102,1103 is associated with each operating electrode 1001,1002 in the sensor array 1100. This individual counter-electrode is connected at an additional connection 801 of the respectively-associated operating circuit 1004,1005. A common ground electrode 1101 is also immersed in the electrolyte 701, which common ground electrode 1101 exhibits a larger surface than each of the operating electrodes 1001,1002 or each of the counter-electrodes 1102,1103. The common ground electrode 1101 is brought to electrical ground potential 705. The sensor array 1100 is thus formed from n sensor arrangements, namely a first sensor arrangement with first operating electrode 1001, first counter-electrode 1102 and first operating circuit 1004, a second sensor arrangement (not shown) with second operating electrode, second counter-electrode and second operating circuit, and so on, through an n-th sensor arrangement with an n-th operating electrode 1002, an n-th counter-electrode 1103 and an n-th operating circuit 1005. In total, n units or sensor arrangements are arranged in a matrix shape, whereby a suitable control electronic (not shown) can be provided in turn.

According to FIG. 11, a counter-electrode with a distinctly reduced surface (in comparison with the common counter-electrode 1003 from FIG. 10) is individually associated with each operating electrode. The potential difference between the counter-electrode and the associated operating electrode of each of the sensor arrangements is kept constant. The operating circuits of the operating electrodes measure the respective occurring current and provided these as a measurement signal to an evaluation circuit (not shown) which is connected at the output 708.

For the case when the reaction volume (meaning the electrolytic analyte 701) of an individual electrochemical system (an individual sensor arrangement) is not electrically insulated (thus a current can flow across additional, for example adjacent electrodes; see in particular FIG. 11, but also FIG. 10), for a stable operation of the sensor arrangement, it must be ensured, by way of control both of the current at the operating electrodes and at the counter-electrodes, that just as much current flows into the respective counter-electrode as is drawn from the operating electrode. This control can ensue by way of a circuit in the associated operating electrode that, on the one hand, adjusts the voltage difference between operating electrode and the counter-electrode to a specific, predeterminable value and simultaneously feeds the current measured at the operating electrode back into the counter-electrode. The latter can, for example, ensue using a suitable current reflector circuit. Since such current reflector circuits can exhibit a slight error (meaning a copy factor slightly deviating from “minus one”), such an error current is either to be eliminated inside the circuit or to be accommodated by an additional ground electrode that is arranged in the reaction volume (compare FIG. 11). However, the error of a current reflector circuit is also frequently negligibly low.

Such a circuit offers advantages in highly-integrated, parallel-operated systems made from operating and counter-electrodes. Local changes of the concentration ratios do not lead to instabilities of the system. Each subsystem of the matrix naturally adjusts the potential difference at the electrodes and ensures that both electrode currents are essentially the same in terms of magnitude. The potential of the electrodes relative to the electrolyte is thus also free and automatically arises, such that local concentration changes are compensated.

### Sensor Arrangement—Fourth Embodiment

In the following, a sensor arrangement 1200 according to a fourth exemplary embodiment of the invention is described with reference to FIG. 12.

The sensor arrangement 1200 resembles the sensor arrangement 800 shown in FIG. 8. In FIG. 12, it shown that the operating electrode 1201 is populated with capture molecules 1202 that are immobilized on the operating electrode 1201. According to FIG. 12, a charge reservoir layer 1204 for provision of electrical charge carriers as needed is provided on a counter-electrode 1203. Inasmuch one can say both that the operating electrode 1201 is suitably functionalized using the capture molecules 1202 and that additionally the counter-electrode 1203 is also functionalized by way of the charge reservoir layer 1204. The charge reservoir layer 1204 can, for example, be realized using a polymer matrix, such that a reducible or oxidizable substance can be provided near the surface of the counter-electrode 1203 dependent on whether oxidations or reductions occur at the operating electrode.

The sensor arrangement 1200 is particularly advantageous when an analysis system should not only be operated quasi-static, but rather when dynamic measurements should be conducted (for example, measurements in which voltage spikes occur or alternating voltage voltammetry). If it is a system in which particles to be detected (for example, DNA half-strands) hybridize with capture molecules 1202 immobilized on the surface of the operating electrode 1201 (for example, in a DNA sensor with electrochemical markers), a comparably large charge quantity can accumulate at the operating electrode 1201 given a match in the shortest time.

In the sensor arrangement 1200, a sufficiently large quantity of convertible material is therefore also immobilized on the counter-electrode 1203 as a charge reservoir layer 1204. If this were not the case, the substance to be converted would first have to diffuse from the solution to the counter-electrode 1203, which would considerably increase the time constant of the system and would not in all cases allow an exact control of the electrode potentials.

In dynamic systems, it is additionally advantageous that the areas of the operating electrode 1201 and of the counter-electrode 1203 do not significantly differ. The double-layer capacity of an electrode increases approximately linearly with the area of the electrode. If the area of the counter-electrode is significantly larger than that of the operating electrode, the coupling of the counter-electrode to the electrolytic analyte 701 is significantly better than that of the operating electrode. In this case, given voltage changes, the electrical charge is primarily stored in the double-layer capacity, and it does not lead to a sufficient degree to the required reactions at the counter-electrode that are necessary for stabilization of the electrode potentials. Given a voltage spike, this would lead to an unwanted shift of the electrolyte potential and thus to uncontrolled reactions at the operating electrode. For the same reasons, an operating electrode too large in comparison with the counter-electrode is disadvantageous for the stability of the system.

Nevertheless, if electrode areas are selected significantly different, this is to be taken into account in the selection of the initial inverse voltage. The shape of the electrodes can in principle be freely selected, however, operating electrode and counter-electrode should be arranged at an optimally small distance from one another. Insofar as the manufacturing process of the sensors allows, interdigital electrodes can be used. In this case, it is ensured that both electrodes are exposed to the same chemical environment and their electrochemical potential relative to the electrolyte is thus approximately the same.

### Sensor Arrangement—Fifth Embodiment

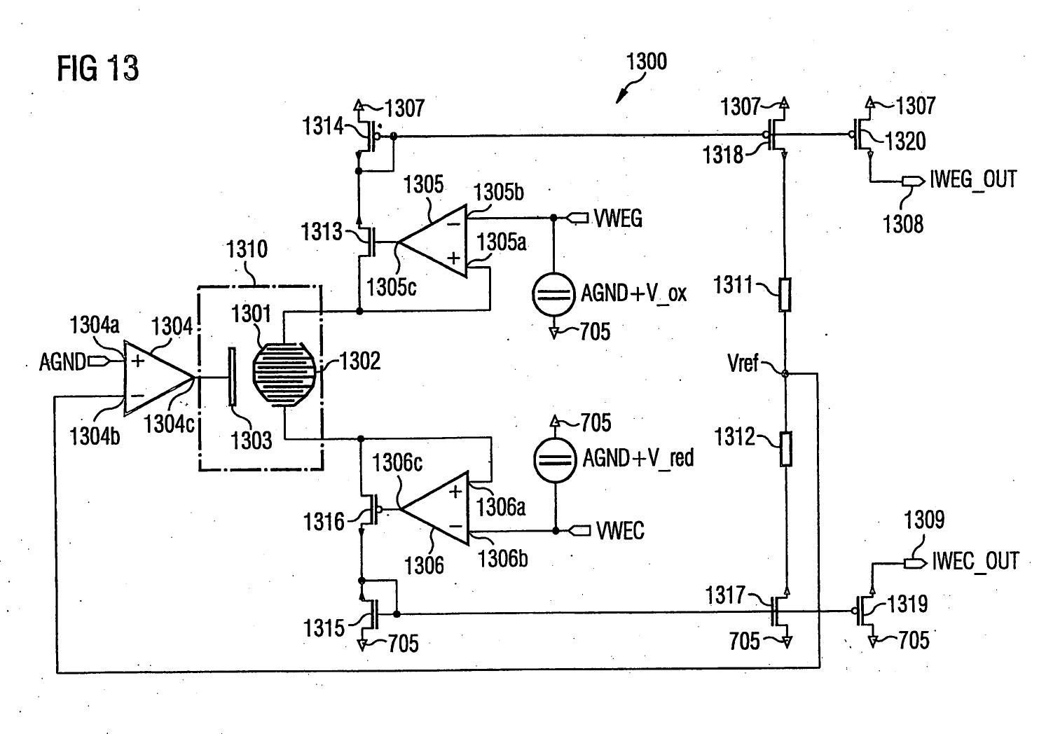

In the following, a sensor arrangement according to a fifth exemplary embodiment of the invention is described with reference to FIG. 13.

The sensor arrangement 1300 comprises an interdigital electrode arrangement made up of a generator electrode 1301 and a collector electrode 1302 that are interlaced with one another like fingers. Apart from the electrodes 1301, 1302, an electrochemical system 1300 comprises a counter-electrode 1303 that, like the electrodes 1301, 1302, is immersed in an electrolytic analyte. The counter-electrode 1303 is coupled with an output 1304c of a first operation amplifier 1304. The generator electrode 1301 is also coupled with a first source/drain connection of a first n-MOS field effect transistor 1313 and is coupled with a non-inverted input 1305a of a second operation amplifier 1305. An inverted input 1305b of the second operation amplifier 1305 is brought to the electrical potential AGND+V_ox.

The collector electrode 1302 is coupled with a first source/drain connection of a second p-MOS field effect transistor 1316 and is coupled with the non-inverted input 1306a of a third operation amplifier 1306. The inverted input 1306b of the third operation amplifier 1306 is brought to the electrical potential AGND+V_red. The output 1305c of the second operation amplifier 1305 is coupled with the gate connection of the first n-MOS field effect transistor 1313. The output 1306c of the third operation amplifier 1306 is also coupled with the gate connection of the second n-MOS field effect transistor 1316.

The second source/drain connection of the first n-MOS field effect transistor 1313 is coupled with a gate connection and with a first source/drain connection of a first n-MOS field effect transistor 1314 whose second source/drain connection is brought to the supply voltage potential 1307.

The gate connection of the first n-MOS field effect transistor 1314 is also coupled with the gate connections of a third n-MOS field effect transistor 1318 and a fourth n-MOS field effect transistor 1320. First source/drain connections of the third and fourth n-MOS field effect transistors 1318,1320 are connected to the supply voltage potential 1307. The second source/drain connection of the fourth n-MOS field effect transistor 1320 is also coupled with a first signal output 1308 for provision of a sensor signal that is characteristic for sensor signals at the generator electrode 1301. The second source/drain connection of the third n-MOS field effect transistor 1318 is also coupled with a first connection of a first ohmic resistor 1311 whose second connection is coupled with a first connection of a second ohmic resistor 1312. The second connection of the second ohmic resistor 1312 is coupled with a first source/drain connection of a third n-MOS field effect transistor 1317 whose second source/drain connection is brought to the electrical ground potential 705.

The gate connection of the third n-MOS field effect transistor 1317 is also coupled with a gate connection and with a first source/drain connection of the second n-MOS field effect transistor 1315 whose second source/drain connection is brought to the electrical ground potential 705. The gate connection of the third n-MOS field effect transistor 1317 is also coupled with a gate connection of a fourth n-MOS field effect transistor 1319. The first source/drain connection of the fourth n-MOS field effect transistor 1319 is also coupled with a second signal output 1309 at which an electrical sensor signal is provided which is characteristic for sensor signals at the collector electrode 1302. The second source/drain connection of the fourth n-MOS field effect transistor 1319 is brought to the electrical ground potential. An electrical node point that is at a potential Vref is arranged between the two ohmic resistors 1311, 1312. This electrical node point is coupled with the inverted input 1304b of the first operation amplifier.

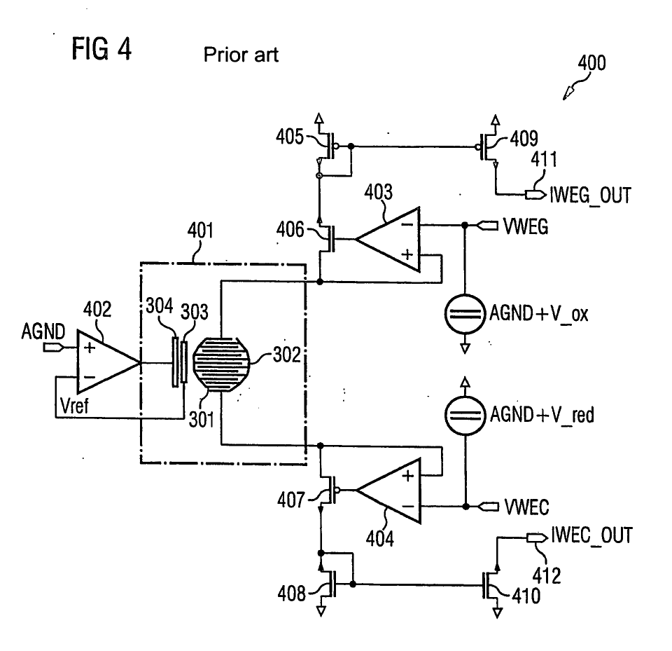

In the circuit of the sensor arrangement 1300, the correct reference potential is clearly derived from a comparison of the electrical currents at both operating electrodes, meaning at the generator electrode 1301 and at the collector electrode 1302. The measurement and regulation circuit for the generator and collector electrodes 1301, 1302 is similar to the circuit according to the prior art shown in FIG. 4.

The currents flowing at the generator electrode 1301 and at the collector electrode 1302 are subtracted from one another in a path using the third n-MOS field effect transistor 1318, the first ohmic resistor 1311 as well as the second ohmic resistor 1312 and the third n-MOS field effect transistor 1317, and the difference is provided at the node Vref. For example, if, in a first operating state, the current at the generator electrode 1301 is larger in terms of magnitude than at the collector electrode 1302, the voltage at node point Vref exceeds the third n-MOS field effect transistor 1318 and the first ohmic resistor 1311. This leads to a decrease of the electrical potential at the counter-electrode 1303. This in turn reduces the generator current. Vref is adjusted to the voltage AGND in equilibrium as it is provided at the non-inverted input 1304a of the first operation amplifier 1304. In contrast to this, if, in a second operating state the current at the generator electrode 1301 is, for example, smaller in terms of magnitude than at the collector electrode, the potential at the counter-electrode 1303 is increased. This in turn increases the generator current.

A reference electrode as required according to the prior art is unnecessary in the sensor arrangement 1300. The measured currents are provides as sensor signals at the first and second signal outputs 1308,1309. It is to be noted that the currents from the signal at generator electrode 1301 or at the collector electrode 1302 lead to two separate sensor signals at the first and second signal outputs 1308, 1309 which, however, contain the same sensor information. The information is inasmuch redundantly available in the sensor arrangement 1300 and can, for example, be averaged, whereby the sensitivity of the sensor arrangement is further increased.

### Sensor Arrangement—Sixth Embodiment

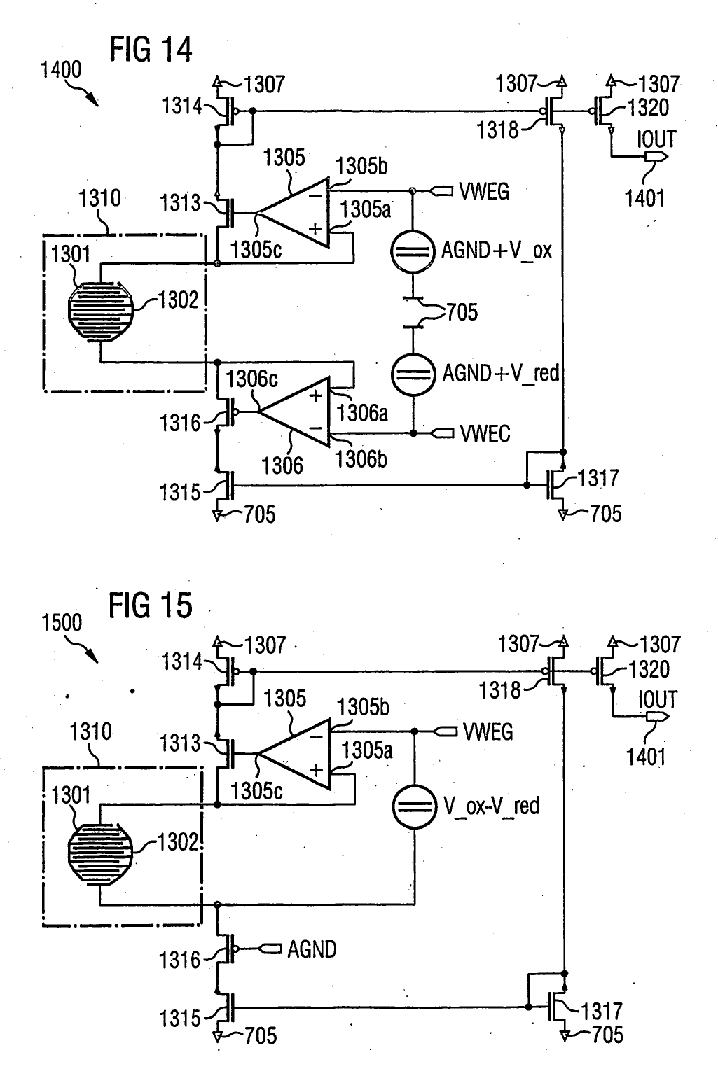

In the following, a sensor arrangement 1400 according to a sixth exemplary embodiment of the invention is described with reference to FIG. 14.

In contrast to the sensor arrangement 1300, in the sensor arrangement 1400, a counter-electrode 1303 is also unnecessary. In the sensor arrangement 1400, the electrochemical system 1310 is therefore formed from the generator electrode 1301, the collector electrode 1302 and the electrolytic analyte in which the electrodes 1301, 1302 are immersed. Since, in the sensor arrangement 1400, a counter-electrode is not provided, the operation amplifier 1304 for activation of the counter-electrode 1303 and the path from FIG. 13 in which the ohmic resistors 1311, 1312 are comprised are also omitted. Furthermore, only a single signal output 1401 is provided instead of the two signal outputs 1308,1309 from FIG. 13.

The potential of the electrolytic analyte clearly automatically arises due to the essentially identical (in terms of magnitude) currents at the generator electrode 1301 and at the collector electrode 1302. In FIG. 14, the potentiostat function is completely integrated into the sensor field. In the sensor arrangement 1400, the branch for detection of the current in the generator electrode 1301 is essentially unchanged relative to FIG. 13. The current measured there is reflected into the branch of the collector electrode 1302 via the third p-MOS field effect transistor 1318, the third n-MOS field effect transistor 1317 and the second n-MOS field effect transistor 1315. The cited components thus clearly serve as a current reflector circuit. It is also to be noted that the gate connection and the source/drain region not situated at ground potential 705 are coupled with one another in the third n-MOS field effect transistor 1317.

The current through the collector electrode 1302 is limited by the described measures and cannot exceed in terms of magnitude the current through the generator electrode.

### Sensor Arrangement—Seventh Embodiment

In the following, a sensor arrangement 1500 according to a seventh exemplary embodiment of the invention is described with reference to FIG. 15.

The sensor arrangement 1500 essentially differs from the sensor arrangement 1400 in that the control circuit (formed from the third operation amplifier 1306) for the voltage regulation at the collector electrode 1302 is replaced by a source follower that is formed from the second n-MOS field effect transistor 1316 connected in the manner shown in FIG. 15. The collector electrode 1302 is coupled with a source/drain connection of the second n-MOS field effect transistor 1316 and is also coupled with the inverted input 1305b of the second operation amplifier 1305. A predeterminable constant potential 1316 is applied at the gate connection of the second n-MOS field effect transistor 1316 connected as a source follower. The voltage difference between the two electrodes 1301,1302 is adjusted via the second operation amplifier 1305.

Apart from the circuit-related simplification, the advantage in the sensor arrangement 1500 is also the fact that only a control amplifier (saving the operation amplifier 1306) operating system is present, and thus stability of the circuit in the control circuit is easy to ensure.

### Sensor Arrangement—Eighth Embodiment

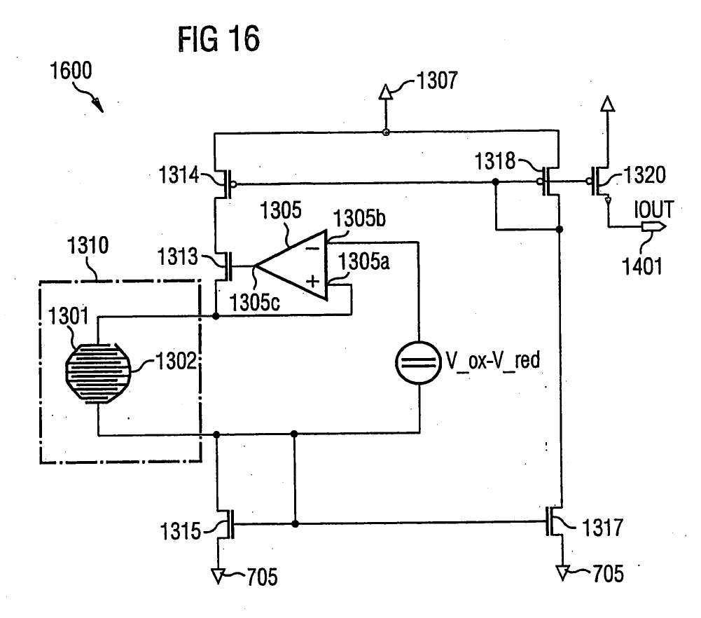

In the following, a sensor arrangement 1600 according to an eighth exemplary embodiment of the invention is described with registration to FIG. 16.

The sensor arrangement 1600 essentially corresponds to the sensor arrangement 1500 and represents a modified exemplary embodiment in which in turn only a single operation amplifier 1305 is required, without the source follower 1316 being required.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of components configured to perform the specified functions. For example, the present invention may employ various integrated circuit components, e.g., memory elements, processing elements, logic elements, which may carry out a variety of functions under the control of one or more microprocessors or other control devices. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Numerous modifications and adaptations will be readily apparent to those skilled in this art without departing from the spirit and scope of the present invention.

### REFERENCE LIST

100 redox cycling sensor arrangement
101 first gold electrode
102 second gold electrode
103 substrate
104 DNA capture molecule
105 analyte
106 first DNA half-strand
107 second DNA half-strand
108 marking
109 secondary molecule
110 reduced molecule
111 oxidized molecule
200 diagram
201 abscissa
202 ordinate
300 interdigital electrode arrangement
301 generator electrode
302 collector electrode
303 reference electrode
304 counter-electrode
305 substrate
306 comparator
307 first amp meter
308 second amp meter
400 sensor arrangement
401 electrochemical system
402 first operation amplifier
403 second operation amplifier
404 third operation amplifier
405 first p-MOS field effect transistor
406 first n-MOS field effect transistor
407 second p-MOS field effect transistor
408 second n-MOS field effect transistor
409 third p-MOS field effect transistor
410 third n-MOS field effect transistor
411 first sensor current output
412 second sensor current output
500 diagram
501 abscissa
502 ordinate
503 electrode current magnitude-electrode voltage curve
504 rest point
505 range of desired reactions
506 range of unwanted reactions
600 diagram
601 abscissa
602 ordinate
603 electrode current magnitude-electrode voltage curve
604 rest point
605 range of first desired reactions
606 range of second desired reactions
607 range of unwanted reactions
700 sensor arrangement
701 electrolytic analyte
702 operating electrode
703 counter-electrode
704 operating circuit
705 ground potential
706 electrode potential device
707 input
708 output
709 insulation device
800 sensor arrangement
801 additional input
900 sensor arrangement
901 ground electrode
1000 sensor array
1001 first operating electrode
1002 n-th operating electrode
1003 common counter-electrode
1004 first operating circuit
1005 n-th operating circuit
1100 sensor array
1101 common mass electrode
1102 first counter-electrode
1103 n-th counter-electrode
1200 sensor arrangement
1201 operating electrode
1202 capture molecule
1203 counter-electrode
1204 charge reservoir layer
1300 sensor arrangement
1301 generator electrode
1302 collector electrode
1303 counter-electrode
1304 first operation amplifier
1304 anon-inverted input
1304 binverted input
1304 coutput
1305 second operation amplifier
1305 anon-inverted input
1305b inverted input
1305c output
1306 third operation amplifier
1306 anon-inverted input
1306b inverted input
1306c output
1307 supply voltage potential
1308 first signal output
1309 second signal output
1310 electrochemical system
1311 first ohmic resistor
1312 second ohmic resistor
1313 first n-MOS field effect transistor
1314 first p-MOS field effect transistor
1315 second n-MOS field effect transistor
1316 second p-MOS field effect transistor
1317 third n-MOS field effect transistor
1318 third p-MOS field effect transistor
1319 fourth n-MOS field effect transistor
1320 fourth p-MOS field effect transistor
1400 sensor arrangement
1401 signal output
1500 sensor arrangement
1600 sensor arrangement

1-28. (canceled)

29. A sensor arrangement for detecting particles that may be contained in an electrolytic analyte, comprising:
an operating electrode that can be electrically coupled with the electrolytic analyte, the operating electrode being arranged such that sensor events occur at the operating electrode given a presence in the sensor arrangement of the electrolytic analyte comprising possible particles to be detected;
an additional electrode that can be electrically coupled with the electrolytic analyte;
an operating circuit coupled with the operating electrode, which operating circuit is arranged such that it adjusts an essentially constant potential difference between the operating electrode and the additional electrode; and
a device that is arranged such that it keeps essentially constant a ratio of electrical currents flowing at the operating electrode and the additional electrode.

30. The sensor arrangement according to claim 29, wherein the electrolytic analyte comprises:
a substance bound to the particles to be detected, which substance possesses a first redox potential in a first concentration in the electrolytic analyte; and
a secondary substance with a second redox potential at a second concentration in the electrolytic analyte, whereby the second concentration is at least as large as the first concentration, and whereby an electrochemical reaction with participation of the substance bound to the particles to be detected ensues given sensor events at the operating electrode.

31. The sensor arrangement according to claim 30, wherein the essentially constant potential difference between the operating electrode and the additional electrode is set to a value that is larger than or equal to a difference between the first redox potential and the second redox potential.

32. The sensor arrangement according to claim 29, wherein the operating circuit comprises an output at which is provided, when there are sensor events, an electrical sensor signal characterizing the sensor events.

33. The sensor arrangement according to claim 29, that is monolithically integrated according to at least one of: a) in a substrate; and b) on a substrate.

34. The sensor arrangement according to claim 29, wherein a first part of sensor arrangement components is provided external from a substrate, and wherein a second part of sensor arrangement components is fashioned according to at least one of: a) in a substrate; and b) on a substrate.

35. The sensor arrangement according to claim 29, wherein the arrangement is arranged as an electrochemical sensor arrangement for detection of at least one of oxidizable and reducible substances.

36. The sensor arrangement according to claim 29, wherein the arrangement is arranged as a biosensor arrangement for detection of biomolecules.

37. The sensor arrangement according to claim 36, wherein the arrangement is arranged for detection of at least one of DNA molecules, oligonucleotides, polypeptides and proteins.

38. The sensor arrangement according to claim 29, wherein capture molecules are immobilized at least at the operating electrode.

39. The sensor arrangement according to claim 36, wherein the arrangement is arranged as a redox cycling sensor arrangement.

40. The sensor arrangement according to claim 36, wherein the arrangement is arranged as a dynamic biosensor arrangement.

41. The sensor arrangement according to claim 29, wherein the operating electrode and the additional electrode comprise a surface of essentially the same size.

42. The sensor arrangement according to claim 29, wherein the device is an electrical circuit.

43. The sensor arrangement according to claim 42, wherein the additional electrode is an additional operating electrode that is arranged such that sensor events occur at the additional operating electrode given the presence in the sensor arrangement of an electrolytic analyte comprising possible particles to be detected.

44. The sensor arrangement according to claim 43, wherein the operating circuit further comprises:
a counter-electrode that can be electrically coupled with an electrolytic analyte, the counter-electrode being arranged such that electrical charge carriers are provided as needed to the electrolyte by way of the counter-electrode, based on a comparison of the electrical currents at the operating electrode and at the additional operating electrode, such that essentially a constant potential difference is set between the operating electrode and the additional operating electrode.

45. The sensor arrangement according to claim 43, wherein the electrical circuit comprises:
a current reflector circuit which is connected such that it provides to the additional operating electrode an electrical current strength, in terms of magnitude, present at the operating electrode.

46. The sensor arrangement according to claim 43, wherein the operating circuit comprises a source follower and precisely one operation amplifier.

47. The sensor arrangement according to claim 29, wherein the device is an insulation device that is arranged such that it electrically insulates the electrolytic analyte, which is electrically coupled with the operating electrode and the additional electrode, from an environment of the electrolytic analyte.

48. The sensor arrangement according to claim 47, wherein the additional electrode is a constant potential electrode that is brought to a constant electrical potential.

49. The sensor arrangement according to claim 47, wherein the additional electrode is coupled with the operating circuit.

50. The sensor arrangement according to claim 47, wherein:
the operating electrode comprises a functionalization, at which functionalization sensor events occur; and
the additional electrode comprises charge carrier reservoir material that, in the case of sensor events, electrical charge carriers are provided at the operating electrode to buffer current surges due to sensor events at the operating electrode.

51. The sensor arrangement according claim 49, further comprising:
a constant potential electrode electrically coupled with the electrolyte, which constant potential electrode is brought to a constant electrical potential.

52. A sensor array comprising a plurality of sensor arrangements according to claim 29.

53. The sensor array according to claim 52, wherein the sensor arrangements are arranged in an essentially matrix-like manner.

54. The sensor array according to claim 52, further comprising:
a control circuit that is arranged for at least one of activation, selection, and readout of a sensor arrangement or of a part of the sensor arrangements.

55. The sensor array according to claim 52, wherein the additional electrode is mutually provided for at least one part of the sensor arrangements and is arranged as a constant potential electrode that is brought to a constant electrical potential.

56. The sensor array according to claim 52, wherein:
in at least one part of the sensor arrangements, the respective additional electrode is coupled with the respective operating circuit, and comprises the one common constant potential electrode that is brought to a constant electrical potential.
